(19) United States
(12) Patent Application Publication
(10) Pub. No.: US 2004/0024345 A1
(43) Pub. Date: Feb. 5, 2004

(54) Glaucoma implant with valveless flow bias

(76) Inventors: Morteza Gharib, San Marino, CA (US); Hosheng Tu, Newport Coast, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/420,354

(22) Filed: Apr. 18, 2003

Related U.S. Application Data
(60) Provisional application No. 60/374,092, filed on Apr. 19, 2002.

(51) Int. Cl. A61F 2/14

(52) U.S. Cl. 604/8; 623/4.1

(57) ABSTRACT

An implant for treating glaucoma in an eye is described, the implant including an inflow portion sized and shaped to fit in the anterior chamber of the eye; an outflow portion sized and shaped fit in at least one of Schlemm's canal, an aqueous collector channel, and an episcleral vein; and a lumen that permits fluid communication from the inflow portion to the outflow portion of the implant, the lumen being configured such that, for a first flow F(i-o) through the lumen in a direction from an inflow end of the implant to an outflow end of the implant, at a first pressure difference P(i-o) between a higher pressure at the inflow end and a lower pressure at the outflow end, said first flow F(i-o) is greater than a second flow F(o-i) through the lumen in a direction from the outflow end to the inflow end, at a second pressure difference P(o-i) between a higher pressure at the outflow end and a lower pressure at the inflow end, where the magnitude of P(o-i) is equal to P(i-o).

### CROSS-REFERENCE TO RELATED APPLICATION

This patent application claims the priority benefit of U.S. Provisional Application No. 60/374,092, entitled “Trabecular Stent Having Valveless Flow Bias Characteristics and Methods of Use,” filed Apr. 19, 2002, the entirety of which is hereby incorporated by reference.

### BACKGROUND OF THE INVENTION

The invention generally relates to methods for reducing pressure in an eye by an implant having a preferential flow direction. More particularly, the invention relates to trabecular stents having valveless flow bias characteristics.

About two percent of people in the United States have glaucoma. Glaucoma is a group of eye diseases that causes pathological changes in the optic disk and corresponding visual field loss, resulting in blindness if untreated. Intraocular pressure elevation is believed to be a major etiologic factor in most cases of glaucoma.

In glaucomas associated with an elevation in eye pressure, the source of resistance to outflow is often in the trabecular meshwork. The tissue of the trabecular meshwork allows aqueous humor (“aqueous”) to enter Schlemm's canal, which then empties into aqueous collector channels in the posterior wall of Schlemm's canal and then into aqueous veins. The aqueous is a transparent liquid that fills the region between the cornea at the front of the eye and the lens. Aqueous humor is constantly secreted by the ciliary body around the lens, so there is a continuous flow of the aqueous humor from the ciliary body to the eye's front chamber. The eye's pressure is determined by a balance between the production of aqueous and its exit through trabecular meshwork (major route) or uveal scleral outflow (minor route). The trabecular meshwork is located between the outer rim of the iris and the back of the cornea. The portion of the trabecular meshwork adjacent to Schlemm's canal causes most of the resistance to aqueous outflow (juxtacanilicular meshwork).

Glaucoma may be grossly classified into two categories: closed-angle glaucoma and open-angle glaucoma. The closed-angle glaucoma is caused by closure of the anterior angle by contact between the iris and the inner surface of the trabecular meshwork. Closure of this anatomical angle prevents normal drainage of aqueous humor from the anterior chamber of the eye. Open-angle glaucoma is any glaucoma in which the angle of the anterior chamber remains open, but the exit of aqueous through the trabecular meshwork and Schlemm's canal is diminished. The exact cause for diminished filtration is unknown for most cases of open-angle glaucoma. However, there are secondary open-angle glaucomas that may involve edema or swelling of the trabecular spaces (from steroid use), abnormal pigment dispersion, or diseases such as hyperthyroidism that produce vascular congestion.

Current therapies for glaucoma are directed at decreasing intraocular pressure. This is initially by medical therapy with eyedrops or pills that reduce the production of aqueous humor or increase the outflow of aqueous. However, these various drug therapies for glaucoma are sometimes associated with significant side effects, such as headache, blurred vision, allergic reactions, death from cardiopulmonary complications, and potential interactions with other drugs. When drug therapy fails, surgical therapy is used. Surgical therapy for open-angle glaucoma consists of laser (trabeculoplasty), trabeculectomy, and aqueous shunting implants after failure of trabeculectomy or if trabeculectomy is unlikely to succeed. Trabeculectomy is a major surgery that is most widely used and is augmented with topically applied anticancer drugs, such as 5-flurouracil or mitomycin-c, to decrease scarring and increase surgical success.

Approximately 100,000 trabeculectomies are performed on Medicare-age patients per year in the United States. This number would increase if the morbidity associated with trabeculectomy could be decreased. The current morbidity associated with trabeculectomy consists of failure (10-15%), infection (a lifelong risk about 2-5%), choroidal hemorrhage (1%, a severe internal hemorrhage from pressure too low resulting in visual loss), cataract formation, and hypotony maculopathy (potentially reversible visual loss from pressure too low).

If one were to bypass the focal resistance to outflow of aqueous at the point of the resistance and use existing outflow mechanisms, surgical morbidity would decrease. The reason for this is that the episcleral aqueous has a backpressure that would prevent the eye pressure from going too low. This would virtually eliminate the risk of hypotony maculopathy and choroidal hemorrhage. Furthermore, visual recovery would be very rapid and risk of infection would be very small (a reduction from 2-5% to about 0.05%). Because of these reasons surgeons have tried for decades to develop a workable surgery for the trabecular meshwork.

The previous techniques that have been tried are trabeculotomy, and other mechanical disruption of the trabecular meshwork, such as trabeculopuncture, goniophotoablation, laser trabecular ablation, and goniocurretage. These are briefly described below.

Goniotomy/Trabeculotomy: Goniotomy and trabeculotomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed secondary to repair mechanisms and a process of “filling in.” The filling in is a detrimental effect of collapsing and closing in of the created opening throughput trabecular meshwork. Once the created openings close, the pressure builds back up and the surgery fails.

Trabeculopuncture: Q-switched Neodymium (Nd):YAG lasers also have been investigated as an optically invasive technique for creating full-thickness holes in trabecular meshwork. However, the relatively small hole created by this trabeculopuncture technique exhibits a filling in effect and fails.

Goniophotoablation/Laser Trabecular Ablation: Goniophotoablation is disclosed by Berlin in U.S. Pat. No. 4,846,172, and describes the use of an excimer laser to treat glaucoma by ablating the trabecular meshwork. This was not demonstrated by clinical trial to succeed. Hill et al. used an Erbium:YAG laser to create full thickness holes through trabecular meshwork (Hill et al., Lasers in Surgery and Medicine 11:341-346, 1991). This technique was investigated in a primate model and a limited human clinical trial at the University of California, Irvine. Although morbidity was zero in both trials, success rates did not warrant further human trials. Failure was from filling in of created defects in trabecular meshwork by repair mechanisms. Neither of these is a viable surgical technique for the treatment of glaucoma.

Goniocurretage: This is an ab interno mechanical disruptive technique. This uses an instrument similar to a cyclodialysis spatula with a microcurrette at the tip. Initial results are similar to trabeculotomy that fails secondary to repair mechanisms and a process of filling in.

Although trabeculectomy is the most commonly performed filtering surgery, Viscocanulostomy (VC) and non-penetrating trabeculectomy (NPT) are two new variations of filtering surgery. These are ab externo (from the outside), major ocular procedures in which Schlemm's canal is surgically exposed by making a large and very deep scleral flap. In the VC procedure, Schlemm's canal is cannulated and viscoelastic substance injected (which dilates Schlemm's canal). In the NPT procedure, the inner wall of Schlemm's canal is stripped off after surgically exposing the canal.

Trabeculectomy, VC, and NPT involve the formation of an opening or hole into the anterior chamber ab externo, under the conjunctiva and scleral flap such that the aqueous humor is drained onto the surface of the eye or into the tissues located within the lateral wall of the eye. These surgical operations are major procedures with significant ocular morbidity. When Trabeculectomy, VC, and NPT were thought to have a low chance for success, a number of implantable drainage devices have been used to ensure that the desired filtration and outflow of aqueous humor through the surgical opening will continue. The risk of placing a glaucoma implant also includes hemorrhage, infection, and postoperative double vision.

All of the above embodiments and variations thereof have numerous disadvantages and moderate success rates. They involve substantial trauma to the eye and require great surgical skill by creating a hole over the full thickness of the sclera/cornea into the subconjunctival space. The procedures are mostly performed in an operating room generating a facility fee and anesthesiologist's professional fee and have a prolonged recovery time for vision. The complications of filtration surgery have inspired ophthalmic surgeons to look at other approaches to lowering intraocular pressure.

The trabecular meshwork and juxtacanilicular tissue together provide the majority of resistance to the outflow of aqueous and, as such, are logical targets for surgical removal in the treatment of open-angle glaucoma. In addition, minimal amounts of tissue are altered and existing physiologic outflow pathways are utilized. Trabecular surgery has the advantage of much lower risk of choroidal hemorrhage and infection, and it uses existing physiologic outflow mechanisms. This surgery could be performed under topical anesthesia in a physician's office with rapid visual recovery in an ab inferno procedure.

To prevent aqueous or blood from refluxing, several devices use a valve mechanism for flow restriction. However, there is a need for a trabecular stent comprising a flow-restricting, valveless mechanism so as to restrict blood or aqueous from flowing back into the anterior chamber of the eye once a drainage trabecular stent is placed. A check valve associated with a tiny trabecular stent for a unidirectional flow pattern may have several potential disadvantages. First, it consists of a moving mechanical component (for example, a valve leaflet) that is difficult to incorporate within a hollow lumen of a trabecular stent as small as 150 microns. Second, a check valve may be relatively bulky and might hinder or obstruct the volumetric flow rate or flow velocity for effectively lowering the intraocular pressure. And lubricating a tiny moving component is a challenge.

Therefore, there is a need for treating glaucoma by using a trabecular stent having a valveless flow bias mechanism for preferentially favoring liquid flow in one direction over the opposite direction.

### SUMMARY OF THE INVENTION

The trabecular bypass surgery and trabecular shunt (also known as a “stent”) used for trabecular bypass surgery disclosed herein may be used in ab interno or ab externo procedures.

Some aspects of the invention include an implant for treating glaucoma in an eye, the implant including an inflow portion sized and shaped to fit in the anterior chamber of the eye; an outflow portion sized and shaped to fit in at least one of Schlemm's canal, an aqueous collector channel, and an episcleral vein; and a lumen that permits fluid communication from the inflow portion to the outflow portion of the implant, the lumen being configured such that (i) a pressure difference AP between a higher pressure at the inflow end and a lower pressure at the outflow end will yield a flow F(i-o) through the lumen in a direction from an inflow end of the implant to an outflow end of the implant, and (ii) a pressure difference of the same magnitude AP between a higher pressure at the outflow end and a lower pressure at the inflow end will yield a flow F(o-i) through the lumen in a direction from the outflow end to the inflow end, said flow F(i-o) being significantly greater than said flow F(o-i).

Certain embodiments further include a material type such as porous material, semi-rigid material, soft material, hydrophilic material, hydrophobic material, hydrogel, elastic material, meshed material, and/or expandable material.

Some embodiments further include a material such as silicone and/or polyurethane.

Certain embodiments further include a material such as polyvinyl alcohol, polyvinyl pyrolidone, collagen, heparinized collagen, tetrafluoroethylene, fluorinated polymer, fluorinated elastomer, flexible fused silica, polyolefin, polyester, polysilicon, stainless steel, titanium, and/or Nitinol.

Some embodiments further include a material such as Teflon, polyimide, hydrogel, heparin, and/or a drug.

In some embodiments, the drug is selected from the group consisting of intraocular pressure-lowering agents, anti-inflammatory agents, anti-angiogenic agents, optic nerve protecting agents, and/or antiproliferative agents.

In some embodiments, an outside diameter of the implant is between about 20 μm and about 500 μm. In certain embodiments, a luminal diameter of the implant is between about 10 μm and about 150 μm. In some embodiments, a length of the lumen is between about 100 μm and about 300 μm.

One aspect of the invention includes an implant for treating glaucoma in an eye, the implant including an inflow portion sized and shaped to fit in the anterior chamber of the eye; an outflow portion sized and shaped fit in at least one of Schlemm's canal, an aqueous collector channel, and an episcleral vein; and a lumen that permits fluid communication from the inflow portion to the outflow portion of the implant, the lumen gradually decreasing in cross-sectional area in a direction extending from the outflow portion to the inflow portion.

Another aspect of the invention includes an implant for treating glaucoma in an eye, the implant including an inflow portion sized and shaped to fit in the anterior chamber of the eye; an outflow portion sized and shaped fit in at least one of Schlemm's canal, an aqueous collector channel, and an episcleral vein; and a lumen that permits fluid communication from the inflow portion to the outflow portion of the implant, the lumen having a taper such that a first cross-sectional area in the outflow portion is greater than a second cross-sectional area of the lumen in the inflow portion.

A further aspect of the invention includes a method of treating glaucoma, including providing an implant having a lumen that permits fluid communication from an inflow end to an outflow end of the implant, the lumen being configured such that a first flow F(i-o) through the lumen in a direction from an inflow end of the implant to an outflow end of the implant at a first pressure difference P(i-o), characterized by a higher pressure at the inflow end than at the outflow end, is greater than a second flow F(o-i) through the lumen in a direction from the outflow end to the inflow end at a second pressure difference P(o-i), characterized by a higher pressure at the outflow than at the inflow end, wherein the magnitude of P(o-i) is equal to P(i-o); and placing the implant into the eye, such that the inflow end of the implant is in the anterior chamber of the eye, and the outflow end of the implant is in at least one of Schlemm's canal, an aqueous collector channel, and an episcleral vein.

In some embodiments, the placing includes inserting the implant into the anterior chamber through a corneal incision. In some embodiments, the placing includes inserting the implant into the eye through a scleral incision.

In one aspect of the invention, an implant is provided having flow bias characteristics including a flow-through construct within the implant, the flow-through construct including a proximal lumen having a uniform proximal cross-sectional area, a proximal opening, and a first flow constriction junction at a distal end of the proximal lumen; a distal lumen having a uniform distal cross-sectional area, a distal opening, and a second flow constriction junction at a proximal end of the distal lumen, the distal cross-sectional area is larger than the proximal cross-sectional area; an elongate middle lumen connecting the first flow construction junction of the proximal lumen and the second flow constriction junction of the distal lumen. The implant further facilitates a pressure differential, wherein the pressure differential is applied to the proximal opening and subsequently to the distal opening causing flow bias characteristics.

It is one object to provide a trabecular stent with flow bias characteristics, wherein “flow bias characteristics” is herein intended to mean a higher volumetric flow rate in one direction than that in the reversed direction when a constant differential pressure is applied in either case. This flow bias characteristic is a unique feature of a preferred design, configured to show a preferential flow in one direction under transient flow conditions and steady-state flow conditions.

The stent implant may be made of biocompatible material, which is typically hollow to allow the flow of aqueous humor from one end to the other end. The material for the stent may be selected from the group consisting of porous material, semi-rigid material, soft material, hydrophilic material, hydrophobic material, hydrogel, elastic material, meshed material, or expandable/retractable material, and the like.

In some aspects, an implant or stent is provided to divert aqueous humor in an eye from an anterior chamber into Schlemm's canal, the stent having flow bias characteristics including a flow-through construct within the stent, the flow-through construct including a proximal lumen having a uniform proximal cross-sectional area, a proximal opening, and a first flow constriction junction at a distal end of the proximal lumen; a distal lumen having a uniform distal cross-sectional area, a distal opening, and a second flow constriction junction at a proximal end of the distal lumen, the distal cross-sectional area is larger than the proximal cross-sectional area; a middle lumen connecting the first and second flow construction junctions, wherein the proximal opening is exposed to the anterior chamber and the distal opening is exposed to Schlemm's canal.

In a further aspect, a method is provided for causing preferential flow bias to divert aqueous humor in an eye from an anterior chamber into Schlemm's canal, including: implanting a trabecular stent having a flow-through construct at a trabecular meshwork of the eye, the flow-through construct including a middle lumen connected with a proximal lumen having a proximal opening and a distal lumen having a distal opening, wherein the proximal lumen has a cross-sectional area smaller than a cross-sectional area for the distal lumen; exposing the proximal opening to the anterior chamber and the distal opening to Schlemm's canal; applying a pressure differential to the proximal opening and subsequently to the distal opening; and using the pressure differential to cause a preferential aqueous flow from the anterior chamber into Schlemm's canal.

### BRIEF DESCRIPTION OF THE DRAWINGS

FIG. 1 is a coronal, cross-sectional view of an eye.

FIG. 2 is a cross-sectional view of an anterior chamber angle of the eye of FIG. 1.

FIG. 3 is a cross-sectional cutaway view of a trabecular implant having flow bias characteristics

FIG. 5 is a velocity magnitude profile under a constant positive pressure in a simulated outflow pattern within a trabecular implant.

FIG. 6 is a corresponding pressure profile under same conditions in FIG. 5.

FIG. 7 is a velocity magnitude profile under a constant negative pressure in a simulated inflow pattern within a trabecular implant.

FIG. 8 is a corresponding pressure profile under same conditions in FIG. 7.

### DETAILED DESCRIPTION OF EXEMPLARY EMBODIMENTS

The exemplary embodiments described below relate to reduction of intraocular pressure in an eye through a surgically implanted stent in trabecular meshwork. While the description sets forth various details, it will be appreciated that the description is illustrative only and should not to be construed in any way as limiting the invention. Furthermore, various applications of the invention and modifications thereto that may occur to those who are skilled in the art are also encompassed by the concepts described below.

In some aspects, an implant is provided having flow bias characteristics. In one embodiment, the implant includes a proximal lumen having a uniform proximal cross-sectional area, a proximal opening, and a first flow constriction junction at a distal end of the proximal lumen, a distal lumen having a uniform distal cross-sectional area, a distal opening, and a second flow constriction junction at a proximal end of the distal lumen, wherein the distal cross-sectional area is larger than the proximal cross-sectional area, an elongate middle lumen connecting the first flow construction junction of the proximal lumen and the second flow constriction junction of the distal lumen. In some embodiments, a pressure differential is applied from the proximal opening to the distal opening causing flow bias characteristics.

The implant with bias flow characteristics is useful in certain medical applications. In one embodiment, the proximal lumen is adapted for exposing to an upstream part of a fluid channel while the distal lumen is adapted for exposing to a downstream part of a fluid channel, wherein the fluid channel is a blood vessel or a body fluid conduit, such as a ureter or urethra. The flow bias characteristics are generally defined by a preferential flow in a first direction from the upstream to the downstream of the fluid channel rather than in the opposite direction.

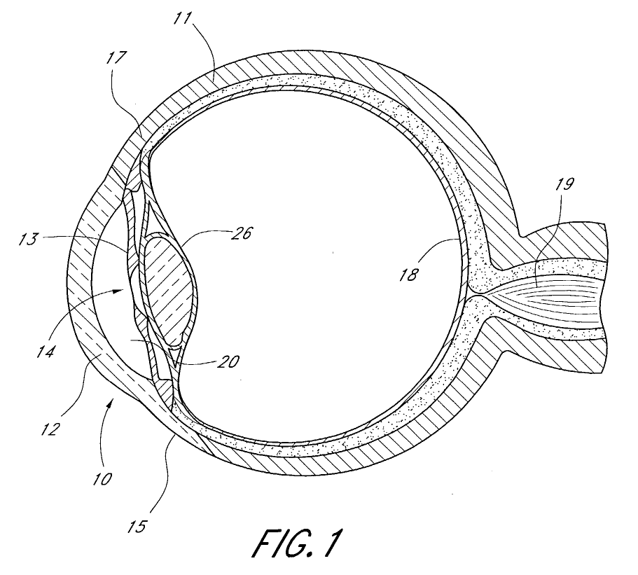

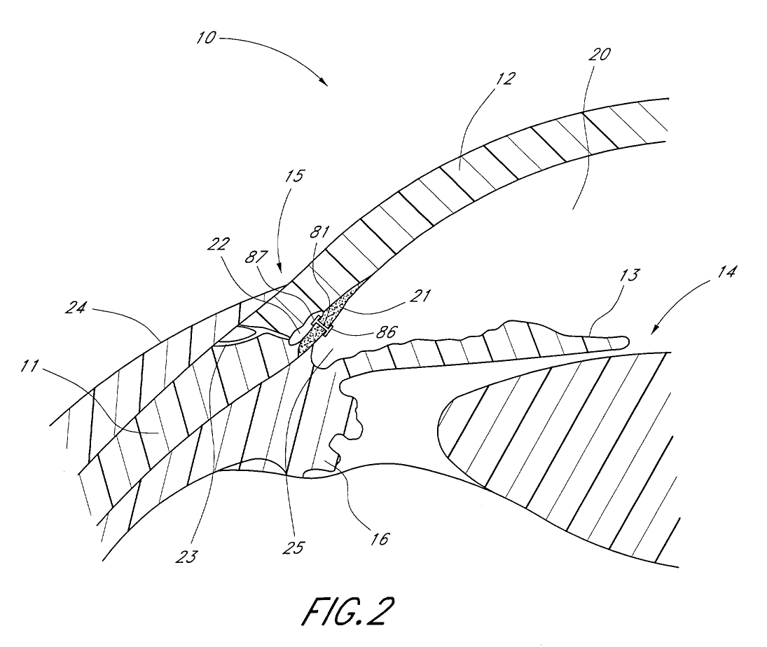

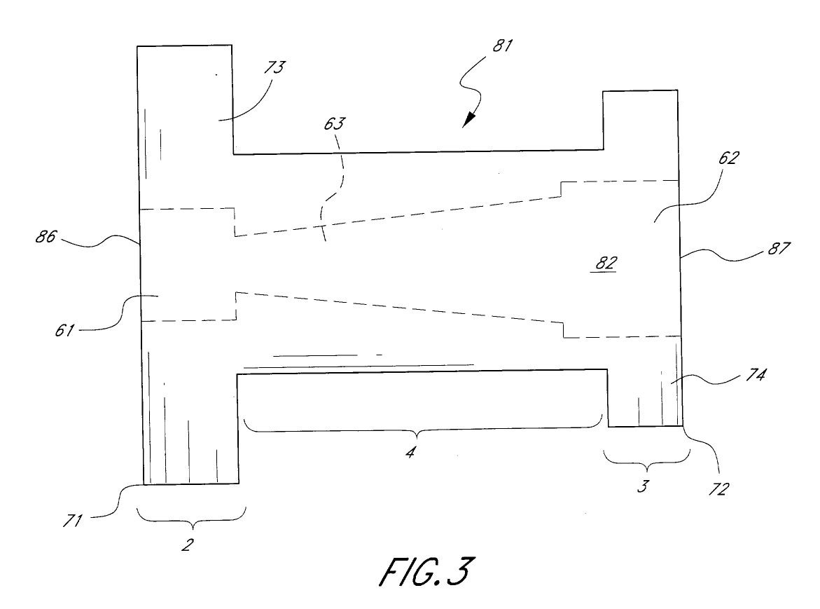

FIG. 1 is a cross-sectional view of an eye 10, while FIG. 2 is a close-up view showing the relative anatomical locations of a trabecular meshwork 21, an anterior chamber 20, and a Schlemm's canal 22. A sclera 11 is a thick collagenous tissue that covers the entire eye 10 except a portion that is covered by a cornea 12. The cornea 12 is a thin transparent tissue that focuses and transmits light into the eye and through a pupil 14, which is a circular hole in the center of an iris 13 (colored portion of the eye). The cornea 12 merges into the sclera 11 at a juncture referred to as the limbus 15. A ciliary body 16 extends along the interior of the sclera 11 and is coextensive with a choroid 17. The choroid 17 is a vascular layer of the eye 10, located between the sclera 11 and a retina 18. An optic nerve 19 transmits visual information to the brain and is the anatomic structure that is progressively destroyed by glaucoma.

The anterior chamber 20 of the eye 10, which is bound anteriorly by the cornea 12 and posteriorly by the iris 13 and a lens 26, is filled with aqueous humor (hereinafter referred to as “aqueous”). Aqueous is produced primarily by the ciliary body 16, then moves anteriorly through the pupil 14 and reaches an anterior chamber angle 25, formed between the iris 13 and the cornea 12. In a normal eye, aqueous is removed from the anterior chamber 20 through the trabecular meshwork 21. Aqueous passes through the trabecular meshwork 21 into Schlemm's canal 22 and thereafter through a plurality of aqueous veins 23, which merge with blood-carrying veins, and into systemic venous circulation. Intraocular pressure is maintained by an intricate balance between secretion and outflow of aqueous in the manner described above. Glaucoma is, in most cases, characterized by an excessive buildup of aqueous in the anterior chamber 20 that leads to an increase in intraocular pressure. Fluids are relatively incompressible, and thus intraocular pressure is distributed relatively uniformly throughout the eye 10.

As shown in FIG. 2, the trabecular meshwork 21 is adjacent a small portion of the sclera 11. Exterior to the sclera 11 is a conjunctiva 24. Traditional procedures that create a hole or opening for implanting a device through the tissues of the conjunctiva 24 and sclera 11 involve extensive surgery, as compared to surgery for implanting a device, as described herein, which ultimately resides entirely within the confines of the sclera 11 and cornea 12. A trabecular stenting device 81 that has an inlet opening 86 and an outlet opening 87 for establishing an outflow pathway, passing through the trabecular meshwork 21, is discussed in greater detail below.

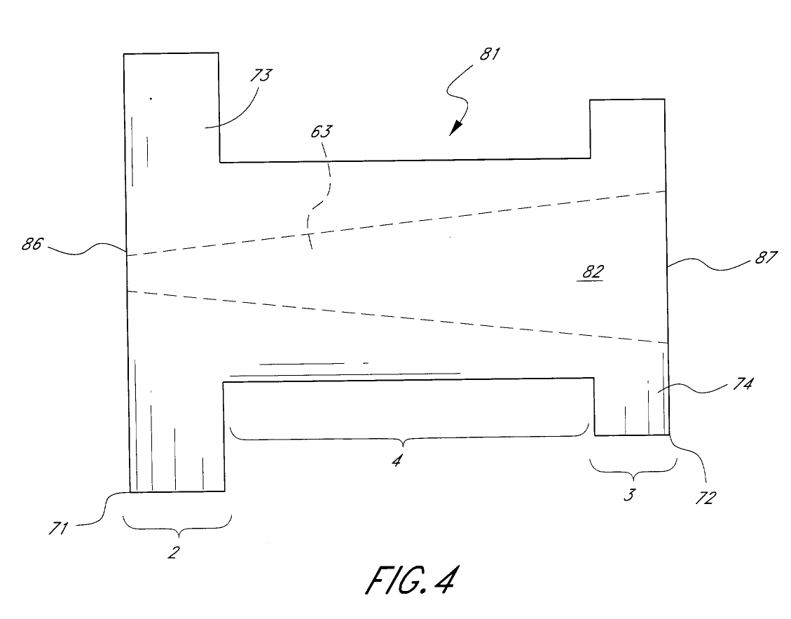

FIG. 3 illustrates a preferred embodiment of a trabecular stenting device 81 that facilitates the outflow of aqueous from the anterior chamber 20 into Schlemm's canal 22, and subsequently into the aqueous collectors and the aqueous veins so that intraocular pressure is reduced. In the illustrated embodiment, the trabecular stenting device 81 comprises a flow-through construct 82 defined by the inlet opening 86, an outlet opening 87 and the lumen surface of the flow conduit. The device has an inlet section 2 with an inlet end 71 and an inlet opening 86, a middle section 4, and an outlet section 3 with an outlet end 72 and an outlet opening 87. The middle section 4 may be an extension of, or may be coextensive with, the inlet section 2. The outlet section 3 is preferably substantially perpendicular to the middle section 4. The flow-through construct 82 of the device 81 further comprises a first, proximal lumen 61 at the inlet section, a second, distal lumen 62 at the outlet section 3 and a third, middle lumen 63 at the middle section 4, wherein all three lumens are connected to each other and are in fluid communication with the inlet opening 86 and the outlet opening 87, thereby facilitating transfer of aqueous through the device 81.

In one preferred embodiment for the flow-through construct 82, the cross-sectional area (or the volume) of the first lumen 61 is smaller than the cross-sectional area (or the volume) of the second lumen 62. The third lumen 63 has a luminal surface within the boundary of the middle section 4, wherein the third lumen is gradually enlarged from the first flow constriction junction 67 located at the distal end of the proximal lumen 61 to the second flow constriction junction 68 that is located at the proximal end of the distal lumen 62. In one embodiment, the interior surface of the middle lumen 63 is shaped as a portion of a cone. As will be apparent to a person skilled in the art, the lumens 61, 62, 63 and the body sections 2, 3, 4 of the stent 81 have a cross-sectional shape that is oval, circular, or other appropriate shape suitably configured for implantation and aqueous transmission.

In some aspects, at least one circumferential ridge or flange 73, 74 is provided at the inlet section 2 and/or at the outlet section 3 to facilitate stabilization of the device 81 once implanted within the eye 10. As disclosed, the inlet section 2 encompasses the inlet lumen 61, the outlet section 3 encompasses the outlet lumen 62, and the middle section 4 encompasses the middle lumen 63. Preferably, the middle section 4, and consequently the middle lumen 63, has a length between the ridges 73 and 74 that is roughly equal to a thickness of the trabecular meshwork 21, which typically ranges between about 100 μm and about 300 μm. In addition, the outlet section 3 may advantageously be formed with a protuberance or spur projecting therefrom so as to further stabilize the device 81 within the eye 10 without undue suturing.

FIG. 4 shows another embodiment of the trabecular stenting device 81. In the illustrated embodiment, the flow-through construct 82 includes a middle lumen 63 that extends from the inlet opening 86 to the outlet opening 87, but there is no distinct proximal lumen 61 or distal lumen 62. The middle lumen 63 gradually decreases in cross-sectional area in a direction extending from the outlet opening 87 to the inlet opening 86. In some embodiments, the middle lumen 63 has a taper, as shown in FIG. 4, such that a first cross-sectional area of the middle lumen 63 near the outlet opening 87 is greater than a second cross-sectional area of the middle lumen 63 near the inlet opening 86.

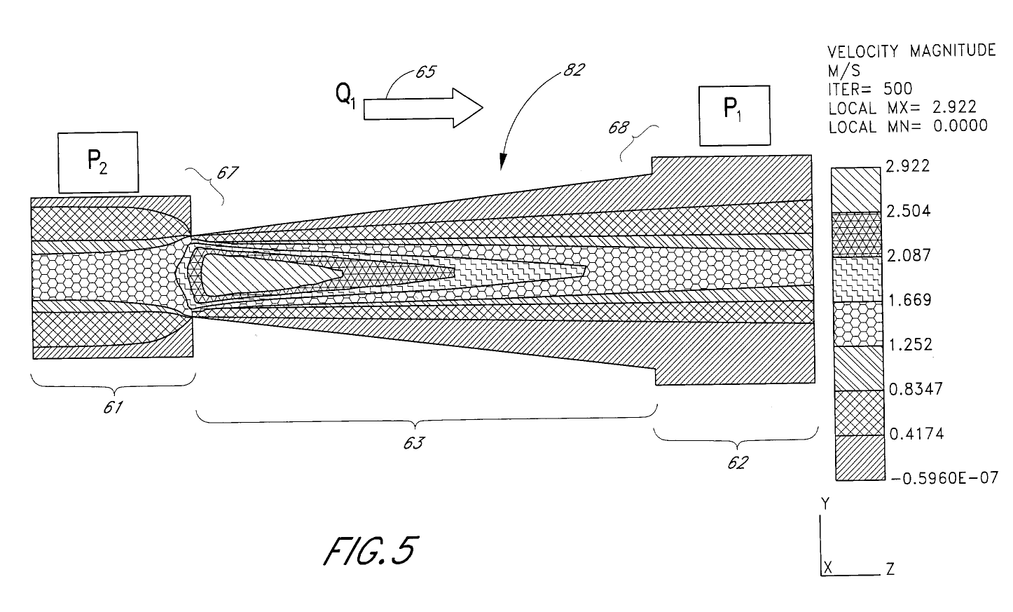

Referring to FIGS. 5 to 8, what is shown is an embodiment for the treatment of glaucoma by a trabecular stent having bias flow characteristics. The stent is usually implanted by a microsurgery means for using a stent implant 81 to bypass diseased trabecular meshwork at the level of trabecular meshwork 21 and use or restore existing outflow pathways. The stent can be implanted in an ab interno procedure through a corneal incision or an ab externo procedure through a scleral incision.

“Trabecular bypass microsurgery” is intended to mean a surgery that creates an access suitably for a stent implantation by means through and bypass the trabecular meshwork. The trabecular microsurgery may comprise an instrument such as a microknife, a hole-saw type applicator, a sharp-end rotator, a pointed guidewire, a sharpened applicator, a sharpened scissor-type cutter, a screw shaped applicator, a retinal pick, an optical fiber, a microcurrette, or the like or combination thereof. The trabecular microsurgery means may further comprise applying thermal energy or cryosurgery in combination with any of the above-mentioned instruments. The thermal energy can be from radiofrequency current, ultrasound current, microwave, laser, infrared or the like.

The stent implant 81 may comprise a biocompatible material, such as medical grade silicone, trade name Silastic™, available from Dow Corning Corporation of Midland, Mich., or polyurethane, trade name Pellethane™, also available from Dow Corning Corporation. In an alternate embodiment, other biocompatible material (biomaterial) may be used, such as polyvinyl alcohol, polyvinyl pyrolidone, collagen, heparinized collagen, tetrafluoroethylene, fluorinated polymer, fluorinated elastomer, flexible fused silica, polyolefin, polyester, polysilicon, stainless steel, titanium, Nitinol, mixture of biocompatible materials, and the like. In a further alternate embodiment, a composite biocompatible material by surface coating the above-mentioned biomaterial may be used, wherein the coating material may be selected from the group consisting of Teflon, polyimide, hydrogel, heparin, therapeutic drugs, and the like. The therapeutic drugs are selected from a group consisting of intraocular pressure (IOP) lowering agents, anti-inflammatory agents, anti-angiogenic agents, optic nerve protecting agents, anti-proliferative agents, and combination thereof.

The main purpose of the stent implant is to assist facilitating the outflow of aqueous in an outward direction into the Schlemm's canal and subsequently into the aqueous collectors and the aqueous veins so that the intraocular pressure is balanced. In some cases, the pressure differential may be reversed instantly or spiked intermittently, it is one object to provide an implant and methods of use that minimize the adversary effects by a preferential bias flow characteristic.

As shown in FIG. 5 to FIG. 8, the stent implant comprises an elongate tubular element having an inlet (or proximal) section 2 having an inlet lumen 61, an outlet (or distal) section 3 having an outlet lumen 62, and a middle section 4 with a middle lumen 63. In an implantation operation, the inlet section 2 is placed within the anterior chamber 20 of an eye 10 while the outlet section 3 is placed within Schlemm's canal 22. Further, a majority or all of the middle section 4 is placed in an opening within trabecular meshwork 21. The distal section may have at least one ridge, rib, or protrusion (not shown) protruding radially outwardly for stabilizing the stent implant inside the existing outflow pathways after implantation. The distal section may also be expandable upon deployment for fixation inside Schlemm's canal. An expandable stent herein may comprise a self-expanding device, heat-activated Nitinol type expanding device, or the like. The outer surface of the device implant 81 is generally biocompatible and tissue compatible so that the interaction/irritation between the outer surface and the surrounding tissue is minimal. The grayish shaded portion in FIGS. 5-8 represents the flow-through construct 82 with a cross-sectional view of the lumen of the stent implant.

In some aspects of the preferred device design as shown in FIGS. 5-8, a flow-through construct 82 is provided comprising a lumen that serves as a preferential one-way flow (also known as “bias flow” herein) controlling means for allowing more aqueous flow in an outflow direction 65 than in a reversed inflow direction 66. The “outflow” herein is meant to indicate an aqueous flow from an anterior chamber 20 of an eye to Schlemm's canal 22, which is a normal flow direction (arrow 65). On the other hand, the “inflow” herein is meant to indicate an aqueous flow from Schlemm's canal 22 of an eye to an anterior chamber 20, which is typically not a normal flow direction (arrow 66). The bias flow controlling means does not include a check valve, a slit valve, a micropump, or any moving component. A semi-permeable membrane and the like may fall under the category of the bias flow mechanism. Other applicable mechanisms by electromagnetic controlling means may also fall under the category of the bias flow mechanism.

As shown in FIG. 3, the stent implant 81 may have a length between about 0.2 mm to over a centimeter, depending on the body cavity this stent implant applies to. The outside diameter of the stent implant may range from about 20 μm to about 500 μm or more. The lumen diameter is preferred in the range between about 10 μm to about 150 μm or more with a special construct as shown in FIGS. 5-8. However, other lumen constructs, sizes or shapes may also be equally applicable.

For positioning the stent 81 to the hole or opening or a virtual opening through the trabecular meshwork (the hole or opening or a virtual opening through the trabecular meshwork is collectively called “access” herein), the stent may be advanced over a guidewire, a fiberoptic (retrograde), or other suitable means. In another embodiment, the stent is directly placed on a delivery applicator and advanced to the implant site, wherein the delivery applicator holds the stent securely during the delivery stage and releases it during the deployment stage after an opening is created using the trabecular microsurgery means as disclosed herein.

In an embodiment of trabecular meshwork surgery, the patient is generally placed in the supine position, prepped, draped, and anesthesia obtained. In one embodiment, a small (about 1 mm or smaller) self-sealing incision is made in the cornea. Through the cornea opposite the stent placement site, an incision is made in trabecular meshwork with an appropriate instrument. The stent 81 is then advanced through the cornea incision across the anterior chamber 20 held in an applicator under gonioscopic (lens) or endoscopic guidance. The applicator is withdrawn and the surgery concluded. The appropriate instrument for creating an incision may be within a size range of 20 to 40 gauge, preferably about 30 gauge.

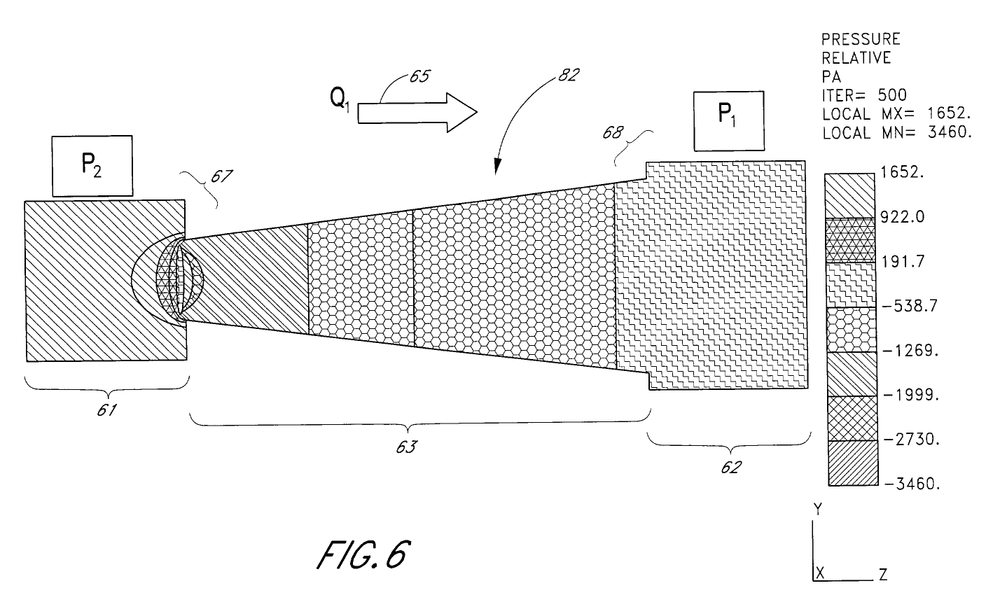

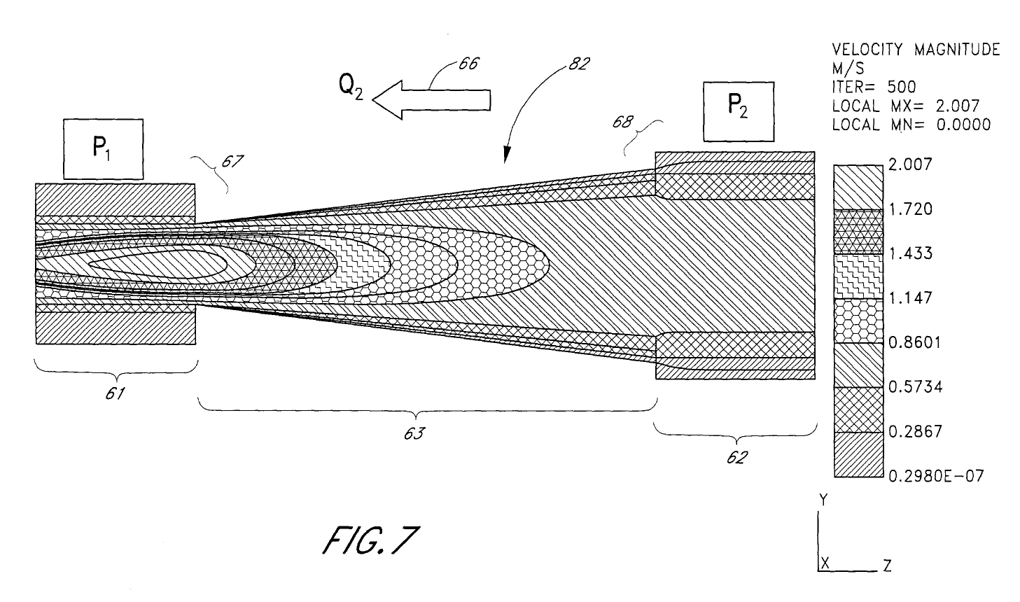

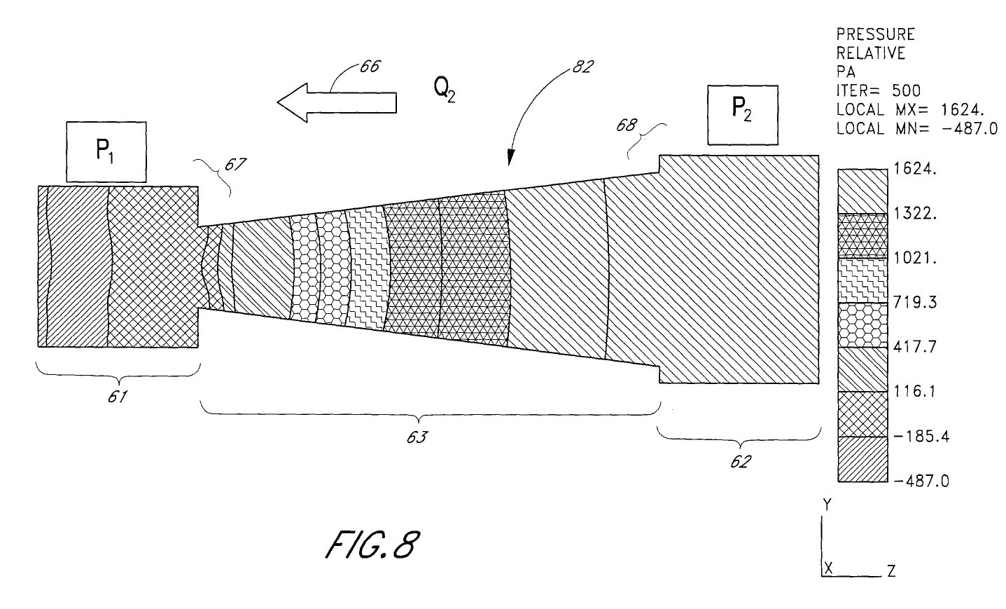

FIG. 5 to FIG. 8 show the liquid flow and pressure pattern from a computer simulation modeling under low N_{Re} laminar flow (wherein the dimensionless Reynolds Number N_{Re}=L×V×ρ÷μ, where L=a characteristic linear dimension of the flow channel, ft; V=linear velocity, ft/sec; ρ=fluid density, 1b/cu ft; μ=fluid viscosity, lb/ft/sec), which is in the ballpark range of a typical aqueous outflow phenomenon from an anterior chamber 20 of an eye through trabecular meshwork 21 out to Schlemm's canal 22. In this particular simulation example, a first pressure of P₁ at 0 Pa and a second pressure of P₂ at 1600 Pa are used. Therefore, the differential pressure applied for either an outflow case (shown in FIGS. 5 and 6) or an inflow case (shown in FIGS. 7 and 8) is identical. The volumetric outflow rate Q₁ and the inflow rate Q₂ are obtained from computer simulations model enabling the bias fluid flowing.

FIG. 5 shows a velocity magnitude profile under a positive constant pressure difference (1600 Pa) from a simulated outflow pattern within a preferred stent design 81. Under a steady-state condition, the high velocity magnitude (darker color) appears at the first flow constriction junction 67 between the inlet lumen 61 and the middle lumen 63; the high velocity magnitude spreads a little downstream towards the outlet lumen 62. Under a low N_{Re} simulation run of the example, there is negligible eddy flow at adjacent to the surrounding surface of the lumens 61, 62, 63. There is almost no difference in velocity magnitude at around the second flow constriction junction 68. In all cases, the aqueous liquid is non-compressible and therefore, the volumetric flow rate at any linear zone axially is constant. The volumetric flow rate is defined mathematically as a product of velocity and its corresponding cross-sectional area.

FIG. 6 shows a corresponding pressure profile under the same conditions as in FIG. 8. A constant high pressure of 1600 Pa covers most of the inlet lumen 61 except at about the first flow constriction junction 67 between the inlet lumen 61 and the middle lumen 63. A very low pressure appears immediately after the first flow constriction junction 67; then the pressure gradually increases downstream towards the outlet lumen 62. In the cases of FIG. 8 and FIG. 8, it simulates the normal physiologic aqueous outflow.

When the differential pressure reverses, such as by squeezing the collecting veins or venting aqueous from an incision in the cornea, a negative pressure between the anterior chamber 20 and Schlemm's canal 22 may exist. This negative pressure phenomenon is represented and shown by FIG. 7 and FIG. 8. FIG. 7 shows a velocity magnitude profile under a negative constant pressure (−1600 Pa) from a simulated inflow pattern within the preferred stent design 81. Under a steady-state condition, the inflow velocity profile is constrained longitudinally. In other words, the high velocity magnitude is limited at around the first flow constriction junction 67; high velocity profiles are shown on both sides of the first flow constriction junction 67. This is in contrast to that shown in the positive differential pressure outflow velocity profile (FIG. 5) where the high velocity is only at the downstream side of the first flow constriction junction 67.

FIG. 8 shows a corresponding pressure profile under the same conditions as in FIG. 7. A constant high pressure of 1600 Pa covers the outlet lumen 62. A very low pressure appears immediately around the first flow constriction junction 67. In the cases of FIG. 7 and FIG. 8, it simulates the abnormal aqueous inflow.

The volumetric flow rate for outflow (arrow 65 in FIG. 5) from the computer simulations modeling is represented by Q₁ while that for inflow (arrow 66 in FIG. 7) is Q₂. The ratio of volumetric flow rates is Q₁/Q₂=1.56. The differential is also expressed as (Q₁−Q₂)/Q₂=36%. This dimensionless differential of 36% in the volumetric flow rate is a basis for “bias flow characteristics.” In other words, a stent 81 of FIG. 3 is sized and configured to exhibit a bias preferential flow rate of 36% in the outflow direction. Other device constructs with various sizes, shapes, and dimensions, such as that in FIG. 4, can yield a bias flow characteristic higher than 36% or lower than 36%. In a transient flow condition, the bias flow characteristics may be different from that number of 36%.

In some aspects of the invention, a trabecular stent is provided to divert aqueous humor in an eye from an anterior chamber into Schlemm's canal, the stent that has flow bias characteristics comprising a flow-through construct within the implant, the flow-through construct comprising: a proximal lumen having a uniform proximal cross-sectional area, a proximal opening, and a first flow constriction junction at a distal end of the proximal lumen; a distal lumen having a uniform distal cross-sectional area, a distal opening, and a second flow constriction junction at a proximal end of the distal lumen, the distal cross-sectional area is larger than the proximal cross-sectional area; and a middle lumen connecting the first flow construction junction of the proximal lumen and the second flow constriction junction of the distal lumen, wherein the proximal opening is exposed to the anterior chamber and the distal opening is exposed to Schlemm's canal.

A trabecular stent having flow bias characteristics is beneficial to alleviate transient or instant negative pressure differential caused by any reason. This reduction in reverse flow (that is, preferential bias flow) is beneficial to operational visualization in the ab interno procedure so as to minimize blood obstruction due to blood backflow into the anterior chamber.

In a preferred aspect, a method is provided for causing preferential flow bias to divert aqueous humor in an eye from an anterior chamber into Schlemm's canal, comprising implanting a trabecular stent having a flow-through construct at a trabecular meshwork of the eye, the flow-through construct comprising an elongate middle lumen connected with a proximal lumen having a proximal opening and a distal lumen having a distal opening, wherein the proximal lumen has a cross-sectional area smaller than a cross-sectional area of the distal lumen; exposing the proximal opening to the anterior chamber and the distal opening to Schlemm's canal; applying a pressure differential to the proximal opening and subsequently to the distal opening; and using the pressure differential to cause a preferential aqueous flow from the anterior chamber into Schlemm's canal. In one embodiment, the step of applying the pressure differential to the proximal opening is by a physiologic aqueous outflow. In another embodiment, the step of applying the pressure differential to the distal opening is by a spiked or surged backflow from Schlemm's canal, wherein the spiked backflow is intermittent or irregular.

From the foregoing description, it should now be appreciated that a novel approach for treating glaucoma with a trabecular stent having a flow bias characteristic has been disclosed for reducing intraocular pressure in an outflow direction preferentially. While the invention has been described with reference to a specific embodiment, the description is illustrative of the invention and is not to be construed as limiting the invention. Various modifications and applications may occur to those who are skilled in the art, without departing from the true spirit and scope of the invention.

What is claimed is:

1. An implant for treating glaucoma in an eye, the implant comprising:
an inflow portion sized and shaped to fit in the anterior chamber of the eye;
an outflow portion sized and shaped to fit in at least one of Schlemm's canal, an aqueous collector channel, and an episcleral vein; and
a lumen that permits fluid communication from the inflow portion to the outflow portion of the implant, the lumen being configured such that (i) a pressure difference AP between a higher pressure at the inflow end and a lower pressure at the outflow end will yield a flow F(i-o) through the lumen in a direction from an inflow end of the implant to an outflow end of the implant, and (ii) a pressure difference of the same magnitude ΔP between a higher pressure at the outflow end and a lower pressure at the inflow end will yield a flow F(o-i) through the lumen in a direction from the outflow end to the inflow end, said flow F(i-o) being significantly greater than said flow F(o-i).

2. The implant of claim 1, further comprising a material type selected from the group consisting of porous material, semi-rigid material, soft material, hydrophilic material, hydrophobic material, hydrogel, elastic material, meshed material, and expandable material.

3. The implant of claim 1, further comprising a material selected from the group consisting of silicone and polyurethane.

4. The implant of claim 1, further comprising a material selected from the group consisting of polyvinyl alcohol, polyvinyl pyrolidone, collagen, heparinized collagen, tetrafluoroethylene, fluorinated polymer, fluorinated elastomer, flexible fused silica, polyolefin, polyester, polysilicon, stainless steel, titanium, and Nitinol.

5. The implant of claim 1, further comprising a material selected from the group consisting of Teflon, polyimide, hydrogel, heparin, and a drug.

6. The implant of claim 5, wherein the drug is selected from the group consisting of intraocular pressure-lowering agents, anti-inflammatory agents, anti-angiogenic agents, optic nerve protecting agents, and antiproliferative agents.

7. The implant of claim 1, wherein an outside diameter of the implant is between about 20 μm and about 500 μm.

8. The implant of claim 1, wherein a luminal diameter of the implant is between about 10 μm and about 150 μm.

9. The implant of claim 1, wherein a length of the lumen is between about 100 μm and about 300 μm.

10. An implant for treating glaucoma in an eye, the implant comprising:
an inflow portion sized and shaped to fit in the anterior chamber of the eye;
an outflow portion sized and shaped fit in at least one of Schlemm's canal, an aqueous collector channel, and an episcleral vein; and
a lumen that permits fluid communication from the inflow portion to the outflow portion of the implant, the lumen gradually decreasing in cross-sectional area in a direction extending from the outflow portion to the inflow portion.

11. An implant for treating glaucoma in an eye, the implant comprising:
an inflow portion sized and shaped to fit in the anterior chamber of the eye;
an outflow portion sized and shaped fit in at least one of Schlemm's canal, an aqueous collector channel, and an episcleral vein; and
a lumen that permits fluid communication from the inflow portion to the outflow portion of the implant, the lumen having a taper such that a first cross-sectional area of the lumen in the outflow portion is greater than a second cross-sectional area of the lumen in the inflow portion.

12. A method of treating glaucoma in an eye, comprising:
providing an implant having a lumen that permits fluid communication from an inflow end to an outflow end of the implant, the lumen being configured such that (i) a pressure difference AP between a higher pressure at the inflow end and a lower pressure at the outflow end will yield a flow F(i-o) through the lumen in a direction from an inflow end of the implant to an outflow end of the implant, and (ii) a pressure difference of the same magnitude ΔP between a higher pressure at the outflow end and a lower pressure at the inflow end will yield a flow F(o-i) through the lumen in a direction from the outflow end to the inflow end, said flow F(i-o) being significantly greater than said flow F(o-i); and
placing the implant into the eye, such that the inflow end of the implant is in the anterior chamber of the eye, and the outflow end of the implant is in at least one of Schlemm's canal, an aqueous collector channel, and an episcleral vein.

13. The method of claim 12, wherein the placing comprises inserting the implant into the anterior chamber through a corneal incision.

14. The method of claim 12, wherein the placing comprises inserting the implant into the eye through a scleral incision.
